(19) United States
(12) Patent Application Publication
(10) Pub. No.: US 2025/0312523 A1
(43) Pub. Date: Oct. 9, 2025

(54) BIOELECTRONIC DEVICE FOR DELIVERY OF SYNTHETIC THERAPEUTICS WITH WIRELESS CONTROL AND METHOD FOR DELIVERING OF THERAPEUTICS USING SUCH DEVICE

(71) Applicant: The Trustees of Columbia University in The City of New York, New York, NY (US)

(72) Inventors: Kenneth L. SHEPARD, Ossining, NY (US); Nanyu ZENG, Troy, NY (US); Yatin GILHOTRA, New York, NY (US); Henry OVERHAUSER, New York, NY (US); Heyu YIN, New York, NY (US)

(73) Assignee: The Trustees of Columbia University in The City of New York, New York, NY (US)

(21) Appl. No.: 19/171,015

(22) Filed: Apr. 4, 2025

Related U.S. Application Data
(60) Provisional application No. 63/575,412, filed on Apr. 5, 2024.

(51) Int. Cl. A61M 1/34; A61K 35/17

(52) U.S. Cl. CPC A61M 1/3489; A61K 35/17; A61M 2202/0445; A61M 2205/3523; A61M 2205/8206; A61M 2230/20

(57) ABSTRACT

An exemplary implantable device and an exemplary method for using the same can be provided. Such exemplary implantable device can comprise a mechanically flexible structure configured for a subdermal implantation, cell reservoirs (i) encased within the mechanically flexible structure, and (ii) configured to house engineered cells for secreting therapeutic peptides therein, integrated circuit (IC) chips including sensing and actuating arrays configured to provide temporal-controlled and dose-controlled release of the peptides, and wireless communication components configured to facilitate a wireless data transmission and a wireless charging of the implantable medical device.

### CROSS REFERENCE TO RELATED APPLICATION(S)

This application relates to and claims priority from U.S. Provisional Patent Application No. 63/575,412, filed on Apr. 5, 2024, the entire disclosure of which is incorporated herein by reference.

### FIELD OF THE DISCLOSURE

The present disclosure relates generally to the technology of synthetic therapeutics, and more particularly, to a bioelectronic device for a delivery of synthetic therapeutics with a wireless control, and to a method for the delivery using such exemplary device.

### BACKGROUND INFORMATION

The persistent rise in obesity and type-2 diabetes mellitus (T2DM) are causing reduced lifespan, health span, and precipitating myriad co-morbidities that, in addition to reducing life quality for many individuals, are inflating health system-wide care costs. [See, e.g., Refs. 1 and 2]. Obesity, by virtue of effects on insulin homeostasis is a major facilitator of type-2 diabetes (T2D) development in individuals predisposed by genetics and lifestyle.

Importantly, even modest weight loss has clinically significant effects on the risks of developing obesity co-morbidities such as diabetes, dyslipidaemia, hypertension, and fatty liver disease. Thus, there is a need to develop safe, effective, and affordable therapies for the treatment of obesity. Despite decades of research detailing the anatomical, physiological, molecular, neuronal, and behavioural mechanisms which contribute to the pathophysiology of obesity and

T2D, there is currently no definitive cure for either disease. As a result, the therapeutic management of these conditions requires lifelong treatment with pharmaceutical and hormone mimetics. [See, e.g., Refs. 3 and 4]. A challenge facing the field is to find the most effective temporally-controlled therapeutics to treat obesity.

Recently, glucagon-like peptide-1 (GLP-1) related peptide therapeutics and GLP-1 derivatives that incorporate the bioactivity of other peptides have emerged as effective treatments for obesity by reducing food intake and weight through physiological satiety pathways. In the search for improved efficacy and additional therapeutic benefits, researchers and drug companies have turned to co-agonists, which commonly engage the GLP-1 receptor along with additional receptors, for example, amylin and glucagon receptors. A major challenge for these treatments is nonadherence due to barriers such as the use of an injected drug, nausea/emesis and associated gastrointestinal side effects. [See, e.g., Ref. 5].

Thus, there is a need to address and/or improve such issues and/or deficiencies which exist in the previous devices, systems, and processes.

### SUMMARY OF EXEMPLARY EMBODIMENTS

The following is intended to be a brief summary of the exemplary embodiments of the present disclosure, and is not intended to limit the scope of the exemplary embodiments of the present disclosure.

In the present disclosure, the above-described challenges can be addressed with an implantable device that reduces adverse events via temporal-and dose-controlled release of peptide satiety signals manufactured by implanted cells. According to the exemplary embodiments of the present disclosure, a mechanically flexible, 1.12 cm×1.84 cm×1.8 mm implantable device can be provided that can be designed and/or configured to be implanted subdermally in an outpatient procedure in the upper arm. The extreme volumetric efficiency of the exemplary embodiment of the device can be effectuated with a simple outpatient procedure. This exemplary device is not limited to provide this indication or these peptides. Indeed, such exemplary device according to the exemplary embodiments of the present disclosure can be used as a reservoir for many different kinds of engineered cells with many possible secreted therapeutics. According to another exemplary embodiments of the present disclosure described herein, it is possible to encase engineered human B-cells and/or β-cells.

Further, an exemplary implantable device can be provided, according to another exemplary embodiments of the present disclosure. Such exemplary implantable device can comprise a mechanically flexible structure configured for a subdermal implantation, cell reservoirs (i) encased within the mechanically flexible structure, and (ii) configured to house engineered cells for secreting therapeutic peptides therein, integrated circuit (IC) chips including sensing and actuating arrays configured to provide temporal-controlled and dose-controlled release of the peptides, and wireless communication components configured to facilitate a wireless data transmission and a wireless charging of the implantable medical device.

For example, the engineered cells can be human B-cells and β-cells. The B cells or the β-cells can be configured to produce a therapeutics under an optical control or an electrical control. The IC chips can include a plurality of on-package antennas configured to facilitate the wireless data transmission and the wireless charging without physical connections. One of the on-package antennas can utilize an On-Off Keying (OOK) radio transceiver that is configured to operate in the Industrial, Scientific, and Medical (ISM) band. Another one of the on-package antennas can be configured to implement the wireless charging.

The implantable device can further comprise 800-μm-thick cavities on either side of the device which can define the cell reservoir volume is defined by 800-μm-thick cavities on either side of the device. The cavities can provide a specific volume for cell housing and ensuring an efficient space utilization.

In another exemplary embodiment of the present disclosure, cavities can be provided on either side of the device which define a cell reservoir volume. The cavities can provide a specific volume for a cell housing and ensuring an efficient space utilization. The cell reservoirs can include filters on at least one side thereof to allow (i) nutrients and oxygen to diffuse into the cell reservoirs, and/or (ii) the therapeutic peptide to diffuse out, and to protect the cells in the reservoir from host immune response. Batteries can be provided which are encapsulated within the mechanically flexible structure. The batteries can be solid-state lithium-ion batteries.

Additionally, the IC chips can be in a direct contact with the cell reservoir. The IC chips can be mounted back-to-back to facilitate access to the cell reservoirs on at least one of the sides of the device. Further, a radio link can be provided between the radio transceiver on the implant and a dongle is connected to a smart phone. The dongle can act as a wireless charger when placed in proximity to the device.

According to another exemplary embodiment of the present disclosure, a method can be provided, using which it is possible to subdermally implant a mechanically flexible device, utilize engineered cells within the mechanically flexible device for producing therapeutic peptides, control a release of the therapeutic peptides using integrated sensing and actuation mechanisms on the mechanically flexible device, and wirelessly control (i) an operation of the device, and (ii) timing and dosage of the release of the peptides.

For example, the release of peptides can be controlled using an application-specific integrated circuit (ASIC) contained within the mechanically flexible device. Such control can thereby facilitate a precise control over a delivery of the therapeutic peptides. In addition, the mechanically flexible device can include a wireless transceiver configured to communicate with an external dongle. Thus, a remote monitoring and an adjustment of delivery parameters of the therapeutic peptides can be facilitated.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a set of illustrations of exemplary designs of an exemplary implantable device, details thereof, and enlarged portion of the device, according to exemplary embodiments of the present disclosure;

FIG. 2 is an illustration of an exemplary design of the exemplary implantable device that includes the ASIC and an interface dongle, according to exemplary embodiments of the present disclosure;

FIG. 3a is a graph of exemplary loop antenna characteristics with S11 as a function of frequency, according to exemplary embodiments of the present disclosure;

FIG. 3b is a graph of exemplary loop antenna characteristics with Directivity plot, according to exemplary embodiments of the present disclosure;

FIG. 4 is an illustration of an exemplary dongle device, according to exemplary embodiments of the present disclosure;

FIG. 5 is an exemplary diagram of a post-processing flow for a two-inch GaN wafers, according to exemplary embodiments of the present disclosure;

FIG. 6 is an spectral plot of a 500-nm-long-pass hybrid absorption/interference emission filter, according to exemplary embodiments of the present disclosure;

FIG. 7 is an illustration of a Monte Carlo simulation of optical intensity through the cell reservoir, according to exemplary embodiments of the present disclosure;

FIG. 8 is an illustration of an exemplary liquid crystal polymer (LCP) package stack- up, according to exemplary embodiments of the present disclosure; and

FIG. 9 is an illustration of an exemplary instruction for performing an exemplary process, according to exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The following is intended to be a description of the exemplary embodiments of the present disclosure, and is not intended to limit the scope of the exemplary embodiments of the present disclosure.

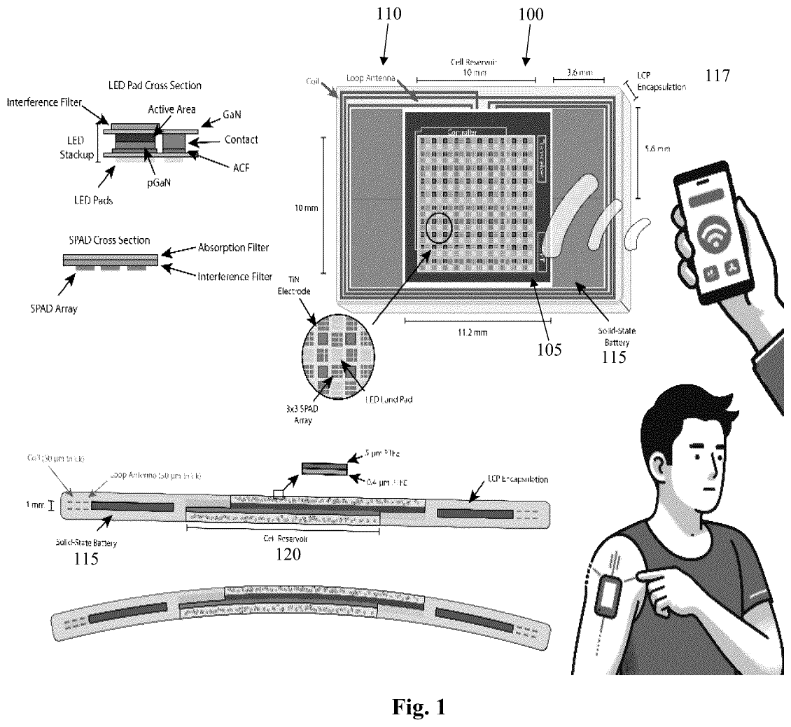

For example, to increase surface-to-volume ratio and facilitate a diffusion as the mechanism for release, an implantable device 100 according to an exemplary embodiment of the present disclosure can have a two-dimensional (2D) form factor, as shown in FIG. 1, and other shapes and/or configurations can be utilized. For example, the exemplary device 100 can include an integrated circuit (IC) chip 105, thinned to about 15 μm, with two such IC chips bonded back-to-back for a total thickness of about 30 μm. The sensing and actuating arrays of each of these IC chips can define the spatial extent of two non-communicating cell reservoirs 120, e.g., each measuring about 10 mm×9 mm. The IC chip 105 can also contain two or more on-package antennas 110, e.g., with at least one antenna for an OOK radio transceiver operating in the ISM band, and at least one other antenna for wireless charging.

For one exemplary embodiment of the implantable device according to the present disclosure, such bonded IC chips can be rotated with respect to each other to position the connections to the batteries on opposite sides of the cell reservoir. The radio transceivers of the two chips can operate at offset carrier frequencies to allow frequency-division multiple access (FDMA) to the two chips. The cell reservoir 120 can be defined by, e.g., about 800-μm-thick cavities on either side of the device, each containing a unique cell type, as described herein.

With the mounting of the PTFE membranes, the well dimensions on each side of the device 100 can be about 10 mm×10 mm×0.7 mm, given the device a total cell reservoir volume of 140 μL. The chip surface in contact with the wells delivers both optical and electrical interfaces to the cells in the same 2D geometry. This can facilitate a dose control by configuring and/or arranging the exemplary implantable device 100 to only target fractions of the cell volume for activation.

For example, the exemplary implantable device 100 can have two banks or more of about 50 20-μA·hr solid-state batteries 115 which are fabricated on silicon substrates at a thickness of 200 μm, giving a total on-implant battery storage of about 2 mA·hr. These exemplary batteries have, e.g., a LiCoO₂ cathode, LiPON ceramic electrolyte and a lithium anode. The entire device can be about 1.8-mm-thick. The periphery of the device containing the batteries is encased in biocompatible liquid crystal polymer (LCP) 117, while the active area of each reservoir can be covered with two PTFE membranes (e.g., 100 μm in thickness each), the outer membrane (e.g., 5 μm pore size) promotes angiogenesis while the inner membrane (0.4 μm pore size) allows for nutrient transport and immune protection similar to other implanted devices. [See, e.g., Ref. 6]. The total implanted volume of the exemplary device 100 can be about 370 μL, making it approximately 38% efficient in use of volume, limited primarily by the requirement for on-chip energy storage.

In one exemplary embodiment of the present disclosure, most or all electronics can be provided on a monolithic integrated circuit measuring, e.g., about 1.12 cm×1.12 cm. Total energy stored in (e.g., four) solid-state lithium-ion batteries can be about 26.6 J; solid-state cells are employed because of their established safety profiles in implantable devices and their high energy density. The custom radio transceiver can consume approximately 40 pJ/bit for both receive and transmit. The receiver can support a wake-up architecture with a stand-by power of about 10 μW, dominated by the power of the crystal oscillator. Wireless powering can deliver about 100 mW of power through the wireless powering coil. This exemplary design can facilitate a complete charging of the device in, e.g., approximately two minutes or less.

### Exemplary Electronics Design: ASIC and Dongle

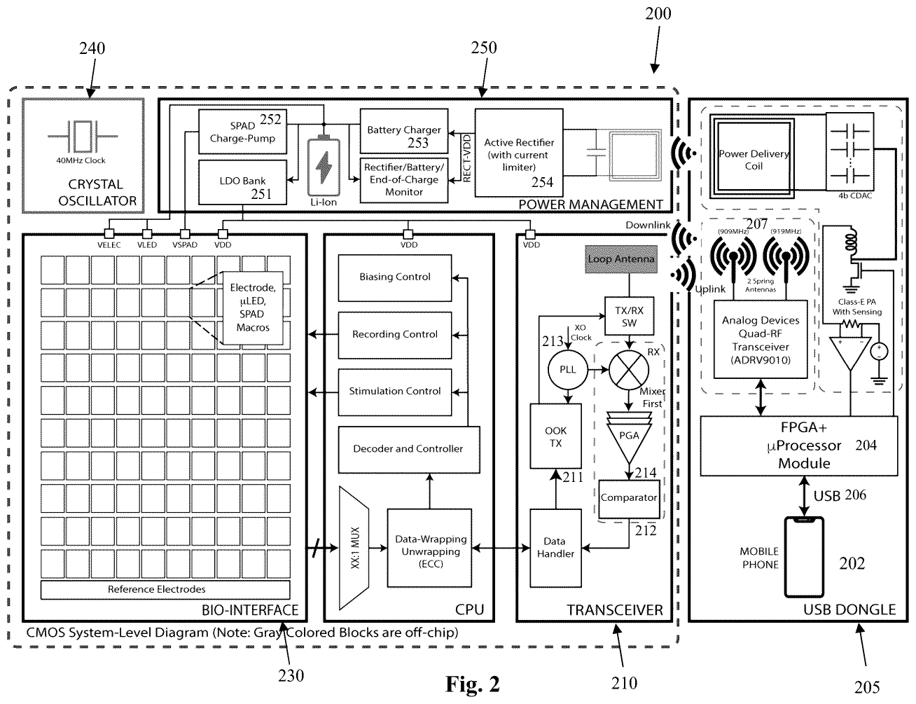

The exemplary designs according to the exemplary embodiments of the present disclosure can utilize a custom application-specific integrated circuit (“ASIC”) for, e.g., all of the electronics. It can be important to reduce the volume of the implant and provide the maximum volumetric efficiency, which can be defined as the ratio of the cell reservoir volume to the total implanted volume of the device. The exemplary schematic diagram of an exemplary implementation of the ASIC 200, e.g., for all of the electronics is shown in FIG. 2. The exemplary electronics of the implant can be all or mostly contained in the ASIC 200. This can be supported by a dongle 205 that can be connected, e.g., with USB-C to an Android smartphone 202. For example, the dongle 205 can communicate wirelessly with the implant and acts as the wireless charging device for the implantable device.

Exemplary Wireless Transceiver. Bluetooth LE (BLE) can often be used as the wireless protocol for implanted devices like this. BLE can be advantageous in that this interface can be natively supported on a smartphone 202. BLE, nonetheless, can be relatively energy inefficient, likely dissipating up to 10 mW when transmitting. This can make BLE difficult to use to achieve a volumetrically efficient, battery-powered design even if a wake-up receiver architecture is chosen. There can also be a considerable overhead in the BLE standard that will be a significant power drain, such as the requirement for advertising.

As a result, according to an exemplary embodiment of the present disclosure, it is possible to instead utilize one or more alternate exemplary radio designs that can be customized for this application. In considering the wireless transceiver design(s), in accordance with various exemplary embodiments of the present disclosure, experiences have been considered in developing radios for low-power implantable and wearable devices, including a custom ultra-wide-band impulse-radio transceiver design [see, e.g., Ref. 8] and/or a frequency-division multiple access (FDMA) radio design [see, e.g., Ref. 9]. To that end, according to the exemplary embodiments of the present disclosure, attributes of both such exemplary designs are combined with an additional constraint that the antenna is implemented within the LCP packaging of the implant, and that a crystal oscillator 240 can be implemented with ultrasmall crystal units (e.g., Kyocera CX1008SB) integrated directly on the integrated circuit.

Thus, the exemplary embodiments of the present disclosure can utilize, e.g., the 902-928 MHz frequency band, which is regulated by the Federal Communications Commission (FCC) Title 47 CFR Part 15 for industrial, scientific, and medical (ISM) proposes. When spread spectrum techniques are not used, the maximum effective isotropic radiated power (EIRP) is limited to −1.23 dBm. For the implantable device, it is possible to estimate a tissue loss (TL) of about 30 dB at 2-cm implantation depth [see, e.g., Ref. 10], about 20-cm free-space path loss (PL) 18 dB, an implant antenna gain (G_{RX}) of about −10 dB, and a transmitter antenna gain (G_{TX}) of about 0 dB. In this exemplary case, the power received at the implant (P_{RX}) when maximum power can be transmitted at the transmitter can be P_{RX}=P_{TX}+G_{TX}−L_{T}−P_{L}+G_{RX}=−59.23 dBm. For a 10-dB link margin, e.g., the wireless receivers on the ASICs can have at least −70 dBm of sensitivity (P_{SEN}). For OOK modulation, the preferred or required SNR for 10⁻³ BER can be about 14 dB. The maximum receiver noise figure can then be, e.g., NF=P_{SEN}−SNR−N₀−BW=30 dB. Since this noise figure requirement or preference can be relaxed, an exemplary mixer-first architecture as shown in FIG. 2 can be used to significantly reduce or eliminate the high-power-consuming RF low-noise amplifier in typical receivers. [See, e.g., Ref. 11].

Exemplary Transceiver Architecture. The exemplary transceiver 210 can include, e.g., a dual-band on-off keying (OOK) transceiver 211/212. The OOK transceiver 211/212 can support up to about 1-Mbps data rate. The 902-928 MHz frequency band can be divided into a lower 908-911 MHz band and a higher 918-921 MHz band. The OOK receivers on the (e.g., two) ASICs can be configured to transmit and receive at about 909 MHz and about 919 MHz, respectively, with approximately 1-MHz bandwidth. The crystal oscillator (XO), described herein, can provide a 40-MHz low-offset reference clock. The transceivers 211/212 (e.g., two transceivers) can operate at the same time using, e.g., frequency-division multi-access (FDMA). [See, e.g., Ref. 9]. In addition to the 1-Mbps normal mode, at least one of the OOK receivers (OOK-RX) 212 can include a low-data-rate mode, which can reduce the receiving data rate to about 1 kbps. Such exemplary OOK transceiver 211/212 can consume about 40 μW in normal operation and about 0.4-μW in low-data-rate mode, and the exemplary XO driver can consume about 10 μW, from a 0.75 V power supply,

According to the exemplary embodiment of the present disclosure, the OOK transmitter 211 and the receiver can share the same on-package antenna through RF switches and time division multiplexing (TDM). When in a standby mode, the OOK transmitter (OOK-TX) 211 can be turned off, and the OOK-RX 212 can be operated in the low-data-rate mode, listening for a wake-up sequence sent from a smartphone 202. In this exemplary mode, the OOK-RX 212 can be duty cycled by, e.g., about 100 times. Once the wake-up sequence is received, the OOK-RX 212 can switch to a normal operating mode. The ASICs can be configured for different sequences to facilitate, e.g., only one of them to wake up should only B-cell or only β-cell activation be required. The wake-up signal can activate the OOK transceiver 211/212. To reduce the power consumption, OOK-RX can be turned off when OOK-TX is transmitting data, and turned back on only when the transmission session is completed. Once a power-off command is received from the OOK-RX 212, the OOK TX 211 can shut off, and the OOK-RX 212 can operate in a low-data-rate mode again.

The baseband OOK-RX 212 can oversample the received data, e.g., by using a clock about four times faster than the data rate, similar to the UART protocol. This can negate or reduce the need for clock-and-date-recovery (CDR) circuits to recover the clock, thereby saving power. The crystal oscillator can ensure a low frequency offset between the ASIC clock and the smartphone clock. For example, a 20-bit wake-up sequence can be used, with the wake-up latency being about 20 ms.

According to the exemplary embodiment of the present disclosure, the OOK-RX can have a low-IF mixer-first architecture. The phase-locked loops (PLLs) 213 on the ASICs can be configured to output, e.g., about 910 MHz and 920 MHz, respectively, from the 40-MHz crystal oscillator. The incoming RF signals can first be down-converted to 1 MHz IF by a passive mixer, and filtered by three stages of 1-MHz band-pass filters (BPFs), thus providing higher than 60 dB isolation between the two bands. The BPFs can also provide about 50 dB of voltage gain. The filtered signal can be fed into an energy detector (ED), composed of a passive self-mixing mixer followed by an integrator. The ED output can then be digitized with a comparator 214 against a predetermined threshold. Since the incoming data is oversampled four times, the integrator in the ED and the comparator runs on a 40 MHz clock. When in the low-data-rate mode, the PLL and BPF should only operate for about 2.5 μsec for every 0.25 msec (4 kHz), resulting in a duty-cycle of about 0.01.

In one example, the OOK-TX 211 can be active only in normal mode. When transmitting a data “1”, the PLL output can be connected to a Class-D RF power amplifier (RF-PA) that can drive the antenna, thus achieving approximately −30 dBm of output power. The RF-PA may not be active when transmitting “0”s, thus saving an average of about 50% power when transmitting long sequences.

Crystal Oscillator Considerations. It is possible to employ ultra-small crystals with dimensions of about 1 mm×0.8 mm 33 0.3 mm. Such crystals can be bump bonded to the CMOS substrate as described below. The steady-state power should be minimized for the XO since the XO is always running. Previous work [see, e.g., Ref. 12] has shown a XO of 39 MHz with 9.2 μW power consumption from 0.7 V power supply can utilize a self-gating technique. It has been suggested that a Colpitts topology, instead of the more common Pierce configuration, can improve power efficiency by a factor of five by leveraging Class-C operation. [See, e.g., Ref. 13]. In accordance with the exemplary embodiments of the present disclosure, a combination of these techniques can be implemented, such as, e.g., a self-gated Colpitts XO. Running at 40 MHz, such exemplary self-gated Colpitts XO can consume approximately 10 μW from a 0.75 V power supply.

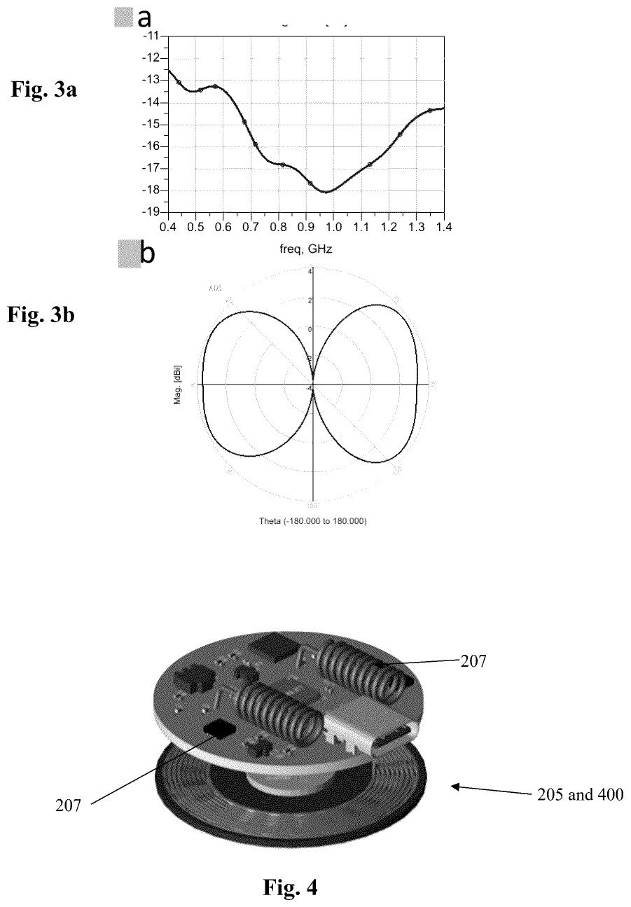

On-package Exemplary Antenna Design. According to the exemplary embodiments of the present disclosure, it is possible to utilize the LCP packaging 117 to integrate larger antennas there for both the radio transceiver and the wireless charging functions as shown topologically in FIG. 1. For example, the loop antennas can be designed as two simple loops stacked, one for each of the two bands, one connecting to one ASIC and the other to the second. The copper traces can be about 10 μm wide and about 50 μm thick. The total length of each antenna can be approximately 35 mm. The exemplary antenna bandwidth is shown in the simulated S11 plot in FIG. 3a which can provide better than −15 dB performance from about 0.7 to 1.25 GHz. The input impedance of the antenna can be about 100 Ω differential. As shown in FIG. 3b, such exemplary antennas can have a maximum directivity of 3 dBi and a radiation efficiency of 0.5%. The presence of nearby metals in the batteries and ASIC can possibly reduce this efficiency in the exemplary implant able device by less than 3 dB.

Exemplary Implant Sensor Array (i.e., BIO-Interface). The exemplary 10-by-10 sensor array (e.g., BIO-Interface) 230 on the IC can have an exemplary pixel structure shown in FIGS. 1 and 2 that can facilitate an electrical stimulation with a TiN electrode, e.g., which can be controlled optical stimulation with a μLEDs, and an optical detection using, e.g., two eight-by-eight arrays of single-photon avalanche diodes (SPADs) fabricated in the CMOS process. These exemplary sensor arrays and their associated fabrication and packaging as described herein Refs. 8 and 14. Such exemplary 2D dimensional geometry can facilitate only portions of the cell population to be stimulated for dose control. Following the fabrication of the ICs in a commercial CMOS foundry, the ICs can be post-processed at the wafer level to deposit the TiN electrodes, passivate the top side of the die, and thin the die to about 15 μm thickness. The TiN electrodes can be be approximately 400 μm×400 μm with a capacitance of about 700 nF. Large counter electrodes can be present on one side of the array, thus facilitating simulation to be monopolar to this to this counter or bipolar through programmable combinations of the on-chip electrodes. Thin-film interference filters combined with absorption filters can be deposited over the SPAD arrays as emission filters to detect fluorescence at a wavelength of about 520 nm. Dies can then be back-to-back bonded. Custom GaN/InGaN μLEDs, spectrally narrowed by a deposited thin-film short-pass excitation filter, can be bonded on each of the multi (e.g., two) sensor array.

For example, the sensor array circuits can support constant-current stimulation pulses with configurable pulse width, pulse rate, pulse amplitude (up to 1 mA), and number of pulses. Upon a depolarization, β-cells display rapid oscillations (primarily due to voltage-gated ion channels, akin to neurons) and slow oscillations (modulated by glucose concentration through Ca²⁺-activated K⁺ ion channels). The electrical stimulation according to the exemplary embodiments of the present disclosure can mimic these patterns of depolarization. The SPAD arrays can be supported by time-gated digital counters which allow photon counts to be collected for fluorescence detection with a dynamic range of about 60 dB. Each μLED can be driven to an optical power density at about 470 nm of up to about 50 mW/cm² with an efficiency of about 10.7% and turn-on voltage of about 2.35 V.

Exemplary Wireless Charging and Power Management. The exemplary batteries on the implantable device 200 can be designed to be charged in approximately two minutes with an exemplary external wireless charger that can also communicate with the implant to monitor charging status. Wireless power transfer (WPT) can rely on, e.g., an inductive coupling at a frequency of about 13.56 MHz to an on-chip coil that is in the periphery of the IC in the two top-level metals. An external charging device can deliver approximately 5 W, about 100 mW of which can be coupled to the implantable device 200 for charging.

To support battery charging, the implant device 200 has an off-chip powering coil with a self-resonance-frequency (SRF) of 13.56 MHz, which is integrated within the LCP packaging as shown in FIG. 1. The AC power received by this coil is converted to DC with an active rectifier 254 [see, e.g., Ref. 8] on the ASIC. Such exemplary rectifier 254, which can have programmable over-voltage protection, can drive the on-chip battery charging unit with more than about 30 mA of current. It is possible to use a linear-regulator-based charger 253.

For example, the charging can begin in a constant-current phase at the maximum charging rate of the solid-state Li-ion batteries with a programmable charging current range from about 20 mA to 1 mA. On-chip battery and rectifier voltage monitoring can be exposed in the instruction set. The dongle 205 can use this to adjust the charging-station transmission power to minimize the difference between the rectifier output voltage and the battery voltage to improve efficiency. At the end of charging, such charging can move from constant-current to constant-voltage with an end-of-charge detector informing the dongle when battery is fully charged. Exemplary on-chip circuits can be provided to disable the connection between the battery and the rectifier 254 when wireless charging is inactive to avoid leakage power consumption. These exemplary techniques can increase the average efficiency of the charger close to be to 90%. [See, e.g., Refs. 15 and 16].

For the normal operation of the device from its on-implant battery, the power-management system 240 can include a low-drop-out (LDO) bank 251 that generated a core 1.5-V supply voltage, which is generally used for the in-pixel counters, the controller of the ASIC, and the OOK transceiver. An exemplary unregulated battery supply (3.7V) can be used to power the μLED Drivers and the current DAC for the electrodes. The exemplary battery supply can also be used as a coarse control for a SPAD charge pump DC-DC converter 252 used to generate the 29-V SPAD voltage, and the regulated supply voltage can be used for a fine control.

Exemplary Dongle design. On the base station (e.g., cell phone), it is possible to support a transceiver dongle which can connect by USB to a smartphone 202. The exemplary dongle can include an exemplary PCB with off-the-shelf components and a power coil. The exemplary PCB can integrate, e.g., two or more 915-MHz spring antennas, one for the lower band and the other for the higher band. The exemplary antennas can connect to the two transceivers, e.g., on an Analog Device ADRV9010. The Analog Device can be controlled by a FPGA and microprocessor module 204 (e.g., SparkFun MicroMod Alorium Sno M2 Processor). Data recovery, synchronization and packet generation logic can also be implemented in the FPGA. The FPGA can interface the USB host on the smartphone through, e.g., a USB Bridge (e.g., FT2233HP). This USB Bridge 206 can also support USB PD which can deliver about 5 V and about 3 A to power up the entire dongle including the coil power amplifier (PA). This Class-E PA drives a powering coil (e.g., Wurth Elektronik 760308101103) position on the bottom side of the dongle, while the PCB on the top side facing up has the spring antennas 207. The exemplary dongle 205 can have a form factor of a cylinder 400, with a diameter of about 35 mm and height of about 20 mm, excluding the USB cable, as shown in FIG. 4.

### Exemplary Post-Processing and Packaging

The exemplary cell wells of either side of implant can be in contact with a sensor and stimulation array that can be fabricated directly on top of the ASIC.

For electrical stimulation of the β cells, TiN electrodes can be fabricated on the chip surface. These electrodes can be augmented by 450-nm blue μLEDs which support an optical power density of up to 50 mW/cm², the spectral leakage from which into the fluorescent band can be controlled by the deposition of a thin-film interference excitation filter. For example, optical detectors in the form of an 8×8 SPAD arrays fabricated directly on the CMOS ASIC can collect fluorescence. The background-rejecting excitation filter can be implemented with a combined interference and absorption filter.

### Exemplary Fabrication and Assembly of the ASIC Chips

Prior to final assembly in the LCP packages, the overall post-processing and fabrication flow can be composed of, e.g., three parts. Wherever possible, this fabrication can be performed on a wafer scale. First, performed wafer-scale on 200-mm CMOS wafers, can be the deposition of the TiN electrodes provided on the top chip surface, substrate thinning, and laser cutting for die singulation. Second, performed on two-inch sapphire wafers, the fabrication of the μLED can be performed which can include the deposition of both interference filters and the laser-lift off setup required for sapphire substrate removal. Third, the GaN μLEDs can be bonded to the thinned CMOS wafer, which is performed at the die level. The last part can be the assembly of the final implant including the PTFE filter and battery integration.

Post-processing of the 200-mm CMOS wafers. The monolithically integrated nature of the wireless implant can be important that can facilitate the entire device to be fabricated with industrial semiconductor manufacturing and packaging processes. After the processing by a commercial CMOS foundry, the wafers can be post-processed. The exemplary base CMOS process can include lead-free bump bonds that can be used to make both the anode and cathode connections to the μLED wafers. Additional solder-bumps near one edge of the ASIC can be used for the connections to the solid-state battery as described herein. Laser dicing is used to singulate the individual implants from the wafer. The top-level polyimide coating is patterned to also avoid covering over the SPAD arrays, which can instead be passivated by the absorption filter, as described herein.

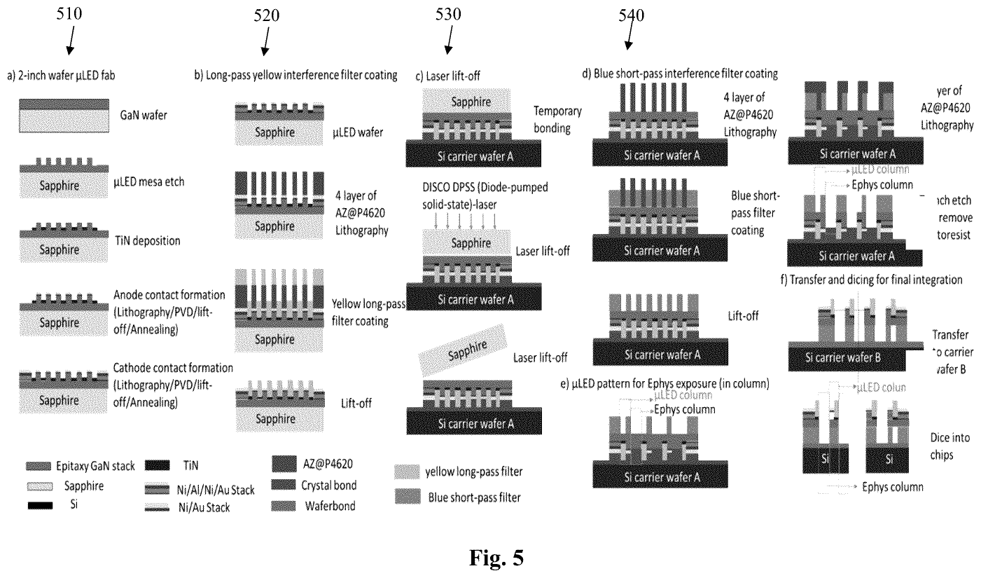

Post-processing of the two-inch GaN wafers. As shown in FIG. 5, the exemplary fabrication of the μLEDs can start with, e.g., two-inch epitaxial multi-quantum-well wafers grown by metalorganic chemical vapor deposition (MOCVD) on sapphire substrates sources from Novagan (procedure 510). This epitaxy can support μLEDs with a peak excitation wavelength of about 450 nm, peak external quantum efficiency (EQE) of about 10.7%, and a turn-on voltage of only about 2.35 V. The exemplary fabrication steps on the μLED GaN wafers can include lithography, mesa RIE etching, metal deposition to define the anode over each μLED, and a cathode “ring” around the entire array. Following by the mesa etch, a 20 nm thick TiN passivation layer was sputtering deposited to eliminate possible shorting between p-doped GaN and n-doped GaN. The p-type Ohmic contacts are formed using a 10-nm-Ni/100-nm-Au deposition, while the n-type contacts can be formed using a 20-nm-Ni/600-nm-Al/100-nm-Ni/400-nm-Au multilayer deposition. Annealing can be performed after each contact deposition in the n-type contact metallization thickness can be chosen to enforce planarity with the p-type contacts. Both contacts can end in Au for solder connection to the CMOS die. For example, approximately 16 complete arrays can be fabricated on each two-inch wafer.

After the μLED fabrication, a long-pass yellow thin-film interference filter can be deposited on the top-side of the wafer, patterned with photoresist for lift-off lithography to facilitate the μLEDs themselves to be clear of the filter (procedure 520). This filter can be approximately 11-30 m, thick and can achieves better then OD 3 rejection of 470 nm at angles of incidence up to 45 degrees, while allowing about 85% transmission at about 520 nm. After this patterned filter deposition, the wafer can be bonded face down on a four-inch silicon carrier wafer using thermal release tape. A laser lift-off process can then be performed by DISCO High-Tec America to remove the sapphire substrate (procedure 530). A blue short pass filter can then be deposited on the wafer, patterned for lift-off lithography to facilitate this filter to be applied only over the μLEDs (procedure 540). This filter can deliver OD 3 rejection at about 520 nm while facilitating greater than 80% transmission at about 465 for angles of incidence up to 30 degrees. A dry etch step can then be performed to make “holes” in the μLED wafer that can be used to expose the underlying TiN electrodes after bonding to the CMOS die. The wafer can then be flipped and transferred to another two-inch silicon carrier wafers using, e.g., WaferBOND HT-10.11 from Brewer Science, and released for the first carrier wafer with heating. The resulting wafer and carrier can then be mechanically diced into individual chips for final assembly.

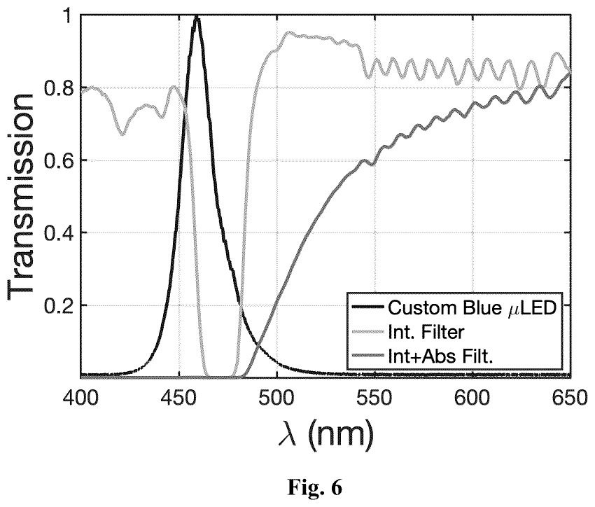

Exemplary Die Level Assembly of the ASIC. For the exemplary final assembly of the ASIC, the thinned CMOS die can be attached face-up to a handle silicon die using WaferBOND HT-10.11. The singulated μLED wafer can then be aligned and flip chip bonded to the CMOS die using the Fintech Lambda tool. After bonding, the space between the μLED wafer and the CMOS die can be underfilled with a custom absorption filter. This absorption filter can be synthesized using a combination of, e.g., 260 mg Valifast Yellow 3150, 50 mg Valifast Green 1501, 400 μL Cyclopentanone, and 200 μL KMPR, After this underfill, e.g., the WaferBOND HT-10.11 connecting the assemble to both handle dice can be dissolved in WaferBOND remover. After release, the entire resulting stack up can be approximately 0.25-mm thick, and can maintain some mechanical flexibility. A radius of curvature of up to 12.5 mm can be accommodated before one risks damaging the assembled ASIC, which is more than sufficient for positioning in the upper arm. The properties of the exemplary resulting hybrid absorption-interference emission filter are provided in the graph that is shown in FIG. 6. For example, an effective OD of six can be achieved for blocking the excitation light at, e.g., 460 nm over the 520-nm green emission.

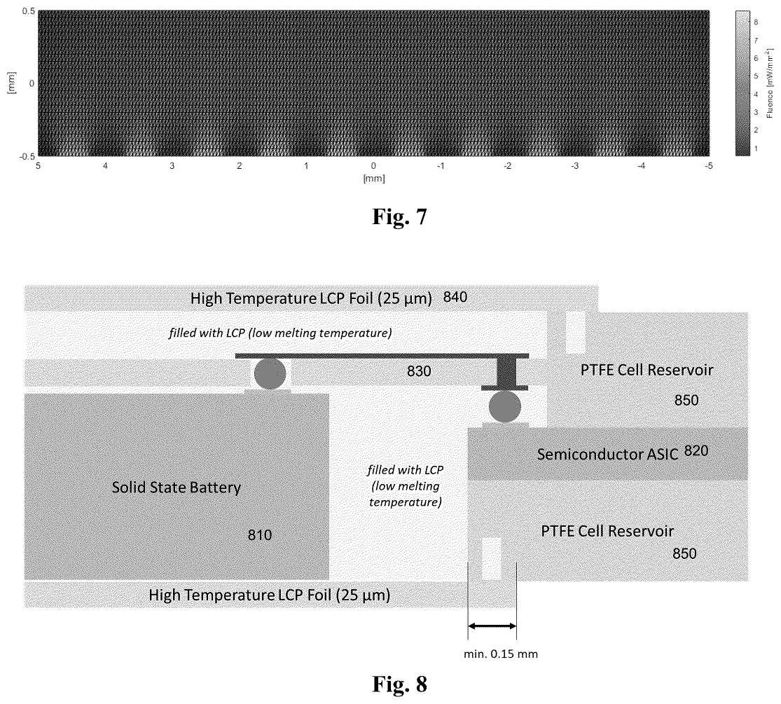

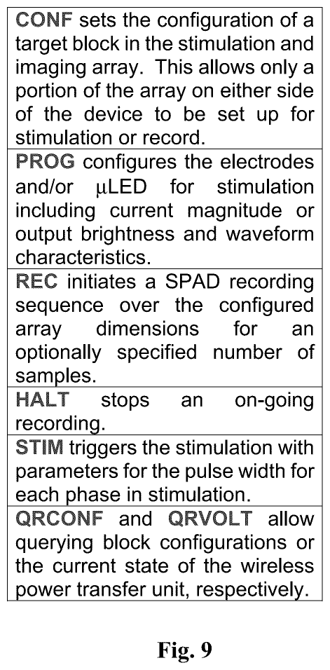

Further, the uniformity of light excitation over, e.g., the 700-μm thickness of the cell volume should be addressed. FIG. 7 shows an exemplary Monte Carlo simulation of the illumination that results from the μLEDs employed here over the extent of the cell reservoir. In these exemplary simulations, the cell matrix can be assumed to have a scattering coefficient of about 3 mm⁻¹, an absorption coefficient of about 0.001 mm⁻¹, an anisotropy of about 0.9, and a refractive index of about 1.36. With the 400-μm-sized μLEDs on a pitch of about 1 mm with an output optical power of about 8.75 mW/cm², illumination at the level of about 2.5 mW/cm² can be achieved over about 80% of the reservoir volume including at the further extents approximately 700 μm away from the surface of the ASIC.

### Liquid-Crystal-Polymer (LCP) Packaging

In the final exemplary device assembly and packaging, two assembled ASICs can be bonded back-to-back using methyl 2-cyanoacrylate adhesive. In this exemplary assembly, the two die can be oriented such that the off-chip connections to the battery are on opposite sides of the two-die stack-up. Interconnections on the LCP packaging can facilitate the two on-chip loop antennas to be formed, as well as the connections between the solid-state batteries 810 and the ASIC 820 (see FIG. 8). The interconnection to the ASIC 820 can be performed using solder-bump connections 830, as shown in FIG. 8. After interconnection encapsulation, the PTFE membranes can be attached to each side and the whole device can be encapsulated by high temperature LCP foil 840. The resulting cell compartment volume can be about 140 μL in volume, comprised of, e.g., 2×70 μL cell chambers, one for each cell type. A cell seeding port for each chamber (e.g., 1.7 mm diameter) can be located at the edge of the cell reservoir 850, 2 mm from the solid-state battery 810. After cell loading, the loading port can be sealed with Dermabond (e.g., Johnson & Johnson product).

### Exemplary Controller Design, Software and Firmware Infrastructure

Exemplary Controller and instruction set. The exemplary chip controller can include, e.g., six main blocks, each implementing a different type of control such as, e.g., command control, electrode array control, wireless power transfer control for battery charging, μLED array control, imaging array control, transmit control, and receiver control. The controller can accept, e.g., eight native instructions, as shown in Fig, 9. Both uplink and downlink packets between the relay station and implant can include error-correcting-code (ECC) bits to facilitate bit errors to be detected and corrected, and a synchronization bits to facilitate the destination to identify when a new packet has been received. The downlink packet can have a preamble field (unused), a three-bit opcode field and an operand field (e.g., 72 bits) that includes a block address and any other parameters. The uplink packet can have a preamble field that can be used to synchronize the order of recorded values during record. For all commands, there can be at least two responses generated, one when the controller starts the execution of the command and a second when the execution is done. In each of the responses, a designated response code can be sent which facilitates the base station to acknowledge the start and end of the execution of a given command.

Exemplary Software Overview. The exemplary software development can be performed with an FPGA development board. The larger FPGA on the development board can facilitate a support of an emulator for the implantable device thus facilitating the software development to proceed without requiring the dongle to be wirelessly connected to the implant. The base station can run the exemplary software with several layers of application program interfaces (APIs) that can provide both a convenient interface to the chip while managing the data transfers. The exemplary programmable logic on the dongle exposes an API to the Android layer that can support the native instructions as well as higher-level functions. The programmable logic can also expose hardware interrupts, which can be used to alert the Android application layer of key events such as the end of a stimulation event or a lost connection. Graphical User Interface (GUI) and interface code development can be performed in Python using Android studio on Linux.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXEMPLARY REFERENCES

The following reference is hereby incorporated by references, in their entireties:
1. Finkelstein, E. A., D. S. Brown, L. A. Wrage, B. T. Allaire, and T. J. Hoerger, Individual and aggregate years-of-life-lost associated with overweight and obesity. Obesity (Silver Spring), 2010. 18(2): p. 333-9.
2. Finkelstein, E. A., J. G. Trogdon, J. W. Cohen, and W. Dietz, Annual medical spending attributable to obesity: payer-and service-specific estimates. Health Aff (Millwood), 2009. 28(5): p. w822-31.
3. Sadry, S. A. and D. J. Drucker, Emerging combinatorial hormone therapies for the treatment of obesity and T2DM. Nat Rev Endocrinol, 2013.
4. Drucker, D. J., S. I. Sherman, R. M. Bergenstal, and J. B. Buse, The safety of incretin-based therapies—review of the scientific evidence. J Clin Endocrinol Metab, 2011. 96(7): p. 2027-31.
5. Moore, P. W., K. Malone, D. VanValkenburg, L. L. Rando, B. C. Williams, H. G. Matejowsky, S. Ahmadzadeh, S. Shekoohi, E. M. Cornett, and A. D. Kaye, GLP-1 Agonists for Weight Loss: Pharmacology and Clinical Implications. Advances in Therapy, 2023. 40(3): p. 723-742.
6. Yang, K., E. D. O'Cearbhaill, S. S. Liu, A. Zhou, G. D. Chitnis, A. E. Hamilos, J. Xu, M. K. S. Verma, J. A. Giraldo, Y. Kudo, E. A. Lee, Y. Lee, R. Pop, R. Langer, D. A. Melton, D. L. Greiner, and J. M. Karp, A therapeutic convection-enhanced macroencapsulation device for enhancing β cell viability and insulin secretion. Proc Natl Acad Sci U S A, 2021. 118(37).
7. Rorsman, P. and F. M. Ashcroft, Pancreatic β-Cell Electrical Activity and Insulin Secretion: Of Mice and Men. Physiol Rev, 2018. 98(1): p. 117-214.
8. Zeng, N., T. Jung, M. Sharma, G. Eichler, F. Fabbri, R. J. Cotton, E. Spinazzi, B. Youngerman, L. Carloni, and K. L. Shepard, A Wireless, Mechanically Flexible, 25□m-Thick, 65,536-Channel Subdural Surface Recording and Stimulating Microelectrode Array with Integrated Antennas, in Symposium on VLSI Circuit. 2023.
9. Chen, C., J. Yang, H. Wang, Z. Cao, S. Kananian, K. Chen, and A. S. Poon. A 90-μW penny-sized 1.2-gram wireless EEG recorder with 12-channel FDMA transmitter for month-long continuous mental health monitoring. in 2022 IEEE Symposium on VLSI Technology and Circuits (VLSI Technology and Circuits). 2022. IEEE.
10. Demir, A. F., Q. H. Abbasi, Z. E. Ankarali, E. Serpedin, and H. Arslan. Numerical characterization of in vivo wireless communication channels. in 2014 IEEE MTT-S International Microwave Workshop Series on RF and Wireless Technologies for Biomedical and Healthcare Applications (IMWS-Bio2014). 2014. IEEE.
11. Wentzloff, D. D., A. Alghaihab, and J. Im. Ultra-low power receivers for IoT applications: A review. in 2020 IEEE Custom Integrated Circuits Conference (CICC). 2020. IEEE.
12. Iguchi, S., T. Sakurai, and M. Takamiya, A low-power CMOS crystal oscillator using a stacked-amplifier architecture. IEEE Journal of Solid-State Circuits, 2017. 52(11): p. 3006-3017.
13. Xu, Z., N. Kimura, K. Okada, and M. Miyahara, Ultralow-power class-c complementary colpitts crystal oscillator. IEEE Solid-State Circuits Letters, 2020. 3: p. 274-277.
14. Pollmann, E. H., H. Yin, I. Uguz, A. Dubey, K. E. Wingel, J. S. Choi, S. Moazeni, Y. Gilhotra, V. A. Pavlovsky, A. Banees, V. Boominathan, J. Robinson, A. Veeraraghavan, V. A. Pieribone, B. Pesaran, and K. L. Shepard, Subdural CMOS optical probe (SCOPe) for bidirectional neural interfacing. bioRxiv, 2023: p. 2023.02.07.527500.
15. Wu, C.-Y., S.-H. Wang, and L.-Y. Tang, CMOS high-efficiency wireless battery charging system with global power control through backward data telemetry for implantable medical devices. IEEE Transactions on Circuits and Systems I: Regular Papers, 2020. 67(12): p. 5624-5635.
16. Pérez, P., F. Veirano, and F. Silveira, A compact lithium-ion battery charger for low-power applications. IEEE Transactions on Circuits and Systems II: Express Briefs, 2022. 69(3): p. 669-673.
17. Gilhotra, Y., H. Overhauser, H. Yin, E. Pollmann, G. Eichler, A. Cheng, T. Jung, N. Zeng, L. Carloni, and K. Shepard. A Wireless Subdural Optical Cortical Interface Device with 768 Co-Packaged Micro-LEDs for Fluorescence Imaging and Optogenetic Stimulation. in IEEE Custom Integrated Circuits Conference. 2024.
18. Moazeni, S., E. H. Pollmann, V. Boominathan, F. A. Cardoso, J. T. Robinson, A. Veeraraghavan, and K. L. Shepard. A Mechanically Flexible Implantable Neural Interface for Computational Imaging and Optogenetic Stimulation over 5.4×5.4 mm 2 FoV. in 2021 IEEE International Solid-State Circuits Conference (ISSCC). 2021. IEEE.

What is claimed is:

1. An implantable medical device, comprising:
a mechanically flexible structure configured for a subdermal implantation;
cell reservoirs (i) encased within the mechanically flexible structure, and (ii) configured to house engineered cells for providing therapeutic peptides therein;
integrated circuit (IC) chips including sensing and actuating arrays configured to provide a temporal-controlled and dose-controlled release of the therapeutic peptides; and
wireless communication components configured to facilitate a wireless data transmission and a wireless charging of the implantable medical device.

2. The device of claim 1, wherein the engineered cells are B-cells or β-cells.

3. The device of claim 2, wherein the B cells or the β-cells are configured to produce a therapeutics under an optical control or an electrical control.

4. The device of claim 1, wherein the IC chips include a plurality of antennas provided in the mechanically flexible structure configured to facilitate the wireless data transmission and the wireless charging without physical connections.

5. The device of claim 4, wherein one of the antennas utilize an On-Off Keying (OOK) radio transceiver that is configured to operate in an Industrial, Scientific, and Medical (ISM) band.

6. The device of claim 5, wherein another one of the antennas is configured to implement the wireless charging.

7. The device of claim 1, further comprising cavities provided on either side of the device which define a cell reservoir volume, wherein the cavities provide a specific volume for a cell housing and ensuring an efficient space utilization.

8. The device of claim 1, wherein the cell reservoirs includes filters on at least one side thereof to allow (i) nutrients and oxygen to diffuse into the cell reservoirs, and (ii) the therapeutic peptide to diffuse out, and to protect the cells in the reservoir from host immune response.

9. The device of claim 1, further comprising batteries which are encapsulated within the mechanically flexible structure.

10. The device of claim 9, wherein the batteries are solid-state lithium-ion batteries.

11. The device of claim 1, wherein the IC chips are in a direct contact with the cell reservoir.

12. The device of claim 1, wherein the IC chips are mounted back-to-back to facilitate access to the cell reservoirs on at least one of the sides of the device.

13. The device of claim 1, wherein a radio link provided between the radio transceiver on the implant and a dongle is connected to a smart phone.

14. The device of claim 13, wherein the dongle acts as a wireless charger when placed in proximity to the device.

15. A method comprising:
implanting a mechanically flexible device subdermally;
utilizing engineered cells within the mechanically flexible device so as to produce therapeutic peptides;
controlling a release of the therapeutic peptides using one or more integrated sensing and actuation mechanisms on the mechanically flexible device; and
wirelessly controlling (i) an operation of the device, and (ii) a timing and a dosage of the release of the therapeutic peptides.

16. The method of claim 15, wherein the release of the therapeutic peptides is controlled using an application-specific integrated circuit (ASIC) contained within the mechanically flexible device, thereby facilitating a substantially precise control over a delivery of the therapeutic peptides.

17. The method of claim 16, wherein the mechanically flexible device includes a wireless transceiver configured to communicate with an external dongle, thereby facilitating a remote monitoring and an adjustment of delivery parameters of the therapeutic peptides.
